(19) United States
(12) Patent Application Publication
(10) Pub. No.: US 2020/0268302 A1
(43) Pub. Date: Aug. 27, 2020

(54) SYSTEMS AND METHODS FOR DIAGNOSING LOWER URINARY TRACT DYSFUNCTION

(71) Applicant: SEOUL NATIONAL UNIVERSITY HOSPITAL, Seoul (KR)

(72) Inventor: Seung-June Oh, Seoul (KR)

(73) Assignee: SEOUL NATIONAL UNIVERSITY HOSPITAL, Seoul (KR)

(21) Appl. No.: 16/566,816

(22) Filed: Sep. 10, 2019

Related U.S. Application Data
(60) Provisional application No. 62/811,050, filed on Feb. 27, 2019.

(51) Int. Cl. A61B 5/20; A61B 5/22; A61B 5/0488; A61B 6/00

(52) U.S. Cl. CPC A61B 5/205; A61B 5/227; A61B 6/5258; A61B 5/204; A61B 6/487; A61B 5/04882

(57) ABSTRACT

A non-transitory tangible computer-readable medium or media include one or more sequences of instructions which, when executed by one or more processors, causes steps to be performed, where the steps include receiving data obtained from baseline clinical evaluation and a urodynamic test for a patient, the diagnostic engine having been trained to associate a data obtained from a urodynamic test with one or more lower urinary tract dysfunction; extracting one or more features from the received data; identifying one or more urodynamic parameters of the patient's lower urinary tract dysfunction, based on the one or more extracted features; and generating an output that includes information of one or more lower urinary tract dysfunction associated with the one or more identified urodynamic parameters.

### CROSS REFERENCE TO RELATED APPLICATIONS

This application claims the benefit of U.S. Provisional Applications No. 62/811,050, entitled “Systems and Methods for Diagnosing Lower Urinary Tract Dysfunction,” filed on Feb. 27, 2019.

### TECHNICAL FIELD

The present invention relates to health care, and more particularly systems and methods for providing computer-aided diagnosis of lower urinary tract dysfunction/disorders based on data obtained from the urodynamic test.

### DESCRIPTION OF THE RELATED ART

Urodynamic study (test) or urodynamics is a study that assesses how the urethral sphincter and bladder are functioning for storing and emptying urine. In general, the urodynamic study seeks to identify various findings associated with a particular disease or condition. Based on the urodynamic study results, a physician(s) can clearly distinguish various types of bladder/urethral dysfunctions in a single disease entity. Typically, a physician performs the urodynamic study to get the detailed information necessary to diagnose the nature of the patient's lower urinary tract dysfunction/disorders, to thereby provide the best treatment option available to the patient. Even if the patients have different entities of the lower urinary tract dysfunction, urodynamic pattern can be similar to those of other entities. Therefore, the prognosis and treatment of the specific lower urinary tract dysfunction of the patient may be similar or identical to those of other disease entities. One of the key roles of the urodynamic study is to determine prognosis and to establish treatment plans in patients with particular lower urinary tract dysfunctions. Thus, the urodynamic study is the single most crucial test in determining the prognosis and treatment of the lower urinary tract dysfunction. Therefore, it is essential for the physicians to accurately interpret urodynamic study results of the patient. Hereinafter, the terms lower urinary tract dysfunction and lower urinary tract disorder are used interchangeably.

Typically, the urodynamic study includes several tests that identify various parameters related to storage and emptying functions of the lower urinary tract, and the physician characterizes the lower urinary tract dysfunction of the patient based on the measured outcomes of the urodynamic parameters. However, the physician's interpretation of urodynamic study results may be affected by several factors, such as the physician's experience in measuring and analyzing the parameters, physician's preferences on the tests, environmental factors, technical errors, physical or psychological condition of the patient, etc. As a consequence, different physicians may arrive at different urodynamic conclusions for the same lower urinary tract dysfunction of the patient, which may result in improper treatments of the lower urinary tract dysfunction of the patient.

In addition, there is a classification system for the current urodynamic study. The International Continence Society (ICS) defines symptoms, signs, urodynamic techniques and urodynamic findings associated with lower urinary tract dysfunction, which are the most widely accepted standardized definitions by the medical community to date. However, physicians still have to interpret and write down the results after the urodynamic tests in person because the ICS does not provide definitions for specific values for normal or abnormal findings in each sub-test. Therefore, in many cases, the final interpretation of urodynamic study results and, thereby, clinical judgments of the physicians may depend on the subjective standards of the physicians.

Recently, artificial intelligence (AI) algorithms have been utilized in various applications to diagnose patient's illnesses. In general, an AI system requires a database for the purpose of training the system, where the database is required to contain a large amount of data that is measured in advance according to standardized test protocols. Currently, due to the lack of sufficient data related to lower urinary tract dysfunction, there is not any AI system that can be applied to diagnose the dysfunction/disorders related to the lower urinary tract of the patient.

The urodynamic study includes several sub-test items, but it is not necessary to perform all of the sub-tests for every patient. For instance, the urodynamic study includes several sub-test items, such as uroflowmetry, filling cystometry, and pressure-flow studies (or equivalently voiding cystometry), where these tests are almost always performed for all patients. The urodynamic study also includes other sub-test items, such as urethral pressure profile, abdominal leak pressure point (ALPP) measurement, and detrusor leak point pressure (DLPP) measurement, where these tests are performed selectively in specific diseases/conditions. Therefore, there is a need for an AI system that is able to provide guidance as to what kind of sub-tests need to be performed for the patients.

Thus, there is a need for systems and methods for training AI algorithms based on a large amount of data from various tests and applying the AI algorithms to correctly characterize the lower urinary tract dysfunction of the patient, to thereby help the physician assess and treat the lower urinary tract dysfunction of the patient.

### SUMMARY OF DISCLOSURE

In one aspect of the invention, a non-transitory tangible computer-readable medium or media include one or more sequences of instructions which, when executed by one or more processors, causes steps to be performed, where the steps includes: receiving data obtained from the functional study of the lower urinary tract for a patient, the diagnostic engine having been trained to associate a data obtained from the functional study of the lower urinary tract with one or more lower urinary tract dysfunction; extracting one or more features from the received data; identifying one or more urodynamic parameters of the patient, based on the one or more extracted features; and generating an output that includes information of one or more lower urinary tract dysfunction associated with the one or more identified urodynamic parameters.

In another aspect of the present invention, a system for characterizing a lower urinary tract dysfunction of a patient includes: one or more processors; and a diagnostic engine communicatively coupled to one or more processors. The diagnostic engine has been trained to associate data obtained from the functional studies of the lower urinary tract with one or more lower urinary tract dysfunction and configured to perform the steps: receiving data obtained from a functional studies of the lower urinary tract for a patient; extracting one or more features from the received data; identifying one or more urodynamic parameters of the patient, based on the one or more extracted features; and generating an output that includes information of one or more lower urinary tract dysfunction of the patient associated with the one or more identified urodynamic parameters.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1A shows an operational diagram of a system for acquiring data of a urinary system according to embodiments of the present disclosure.

FIG. 1B shows a schematic diagram of the urodynamic study 100 for acquiring urodynamic data of a lower urinary tract system according to embodiments of the present disclosure.

FIG. 1C shows a schematic diagram of the pressure measurement system according to embodiments of the present disclosure.

FIGS. 2A and 2B show exemplary data from uroflowmetry for two different patients, respectively, according to embodiments of the present disclosure.

FIG. 3A shows an exemplary data from cystometry during both filling and voiding phase according to embodiment of the present disclosure.

FIG. 3B shows an exemplary data taken during the storage phase of cystometry according to embodiments of the present disclosure.

FIG. 3C shows an exemplary data from cystometry during both filling and voiding phase according to embodiments of the present disclosure.

FIG. 4 shows an exemplary data from cystometry during both filling and voiding phase according to embodiments of the present disclosure.

FIG. 5 shows an exemplary data taken during the voiding phase of cystometry according to embodiments of the present disclosure.

FIG. 6 shows a plot of detrusor pressure as a function of urine flow rate according to embodiments of the present disclosure.

FIG. 7 shows an exemplary data from cystometry (where that of the voiding phase corresponds to the pressure-flow study) according to embodiments of the present disclosure.

FIG. 8 shows an exemplary data taken during a urethral pressure profile measurement according to embodiments of the present disclosure.

FIG. 9 shows an exemplary data from an abdominal leak point pressure measurement according to embodiments of the present disclosure.

FIG. 10 shows a table of urodynamic conclusion according to the International Continence Society (ICS) classification.

FIG. 11 shows a schematic diagram of a system for diagnosing lower urinary tract dysfunction according to embodiments of the present disclosure.

FIG. 12 shows a schematic diagram of a data acquisition station according to embodiments of the present disclosure.

FIG. 13 shows a flowchart illustrating an exemplary process for operating a diagnostic engine according to various embodiments of the present disclosure.

FIG. 14 shows a computer system according to embodiments of the present disclosure.

### DETAILED DESCRIPTION OF THE PREFERRED EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium.

Components shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled” “connected” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. The appearances of the phrases “in one embodiment,” “in an embodiment,” or “in embodiments” in various places in the specification are not necessarily all referring to the same embodiment or embodiments.

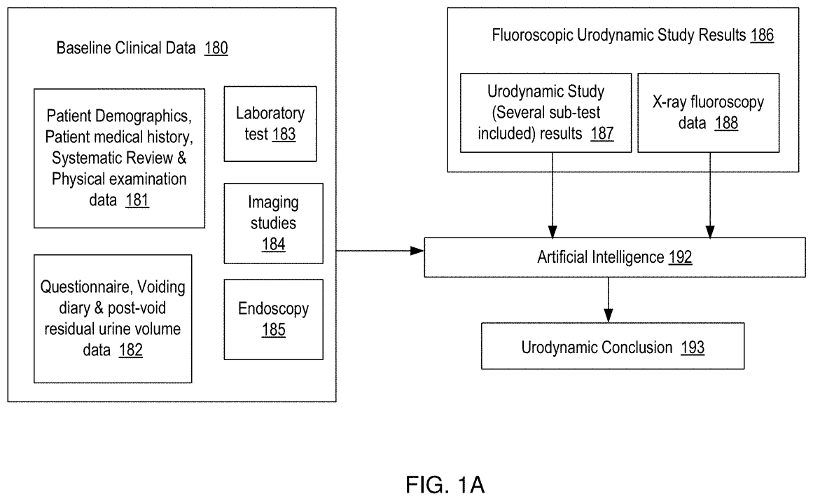

FIG. 1A shows an operational diagram of a system for acquiring data of a urinary system according to embodiments of the present disclosure. As depicted, the artificial intelligence (AI) 192 may characterize the nature of a specific lower urinary tract dysfunction of the patients with single or multiple lower urinary tract dysfunction/disorders so as to draw the urodynamic conclusion. The AI 192 may use data that includes baseline clinical data 180 and fluoroscopic urodynamic study results 186.

In embodiments, the fluoroscopic urodynamic study results 186 may urodynamic study (several sub-test included) results 187 and X-ray fluoroscopy data 188. In embodiments, the AI 192 may perform auto-detection and screening of errors/artifacts in the fluoroscopic urodynamic study results 186 so that the errors/artifacts are screened and eliminated before the interpretation of the results. In embodiments, the detected errors/artifacts may be listed up as an addendum of the urodynamic conclusion 193 of the specific patient.

In embodiments, the baseline clinical data 180 may include various patient clinical data; patient demographics, patient medical history, data obtained by systematic review and physical examination 181; questionnaire, voiding diary and post-void residual (PVR) urine volume data 182; laboratory test results 183; data from imaging studies 184; and endoscopy data 185.

In embodiments, the patient demographics may include gender, age, height, weight, body mass index, abdominal circumference, so on. In embodiments, the patient medical history may include past medical or surgical history, medication history, and current medication. In embodiments, the patient medical history may also include any previous neurological diseases, previous surgical history especially in the brain, spinal cord, vertebrae, genitourinary system or pelvic cavity, which may significantly affect the function of the lower urinary tract. In embodiments, the data obtained from the systemic review may include erectile function and bowel function. In embodiments, the information from physical examination may include motor or sensory function of the trunk, and limbs, lower abdomen, genitourinary area or prostate (presence or absence of prostate enlargement, genitourinary skin eczema due to urinary incontinence, or sacral skin defect), and focused neurological examination. In embodiments, the findings from a focused neurological examination may include perineal sensation to light touch and/or pinprick test, degree of anal tone, presence or absence of bulbocavernosus reflex, voluntary contraction of the anal sphincter, and fecal impaction.

In embodiments, the questionnaire may include the patient's response to the structured questionnaire related to the lower urinary tract symptoms. The questionnaire is a very important tool for quantifying the patient's subjective symptoms because it can assess the severity of lower urinary tract symptoms and its impact on the quality of life of a specific patient.

In embodiments, the quantitative information obtained from voiding diary or frequency-volume chart may include frequency of urination during daytime or night time, interval of voiding, number of urgency or urinary incontinence, average or maximum voiding volume. In embodiments, the PVR urine volume may be assessed by either ultrasound device or urethral catheterization. In embodiments, the laboratory tests may include urine culture of microorganisms, serum creatinine level showing the degree of kidney insufficiency. In embodiments, the anatomical evaluation of the urinary tract may include imaging studies of the kidney and urinary bladder. Urinary tract CT and/or ultrasonic imaging may show damaged kidney(s) or degree of bladder trabeculation suggesting compensatory hypertrophy of the bladder wall secondary to lower urinary tract dysfunction. In embodiments, the cystourethroscopy or endoscopy of the bladder and urethra may identify the presence or absence of urethral stricture, presence or absence of anatomical bladder outlet obstruction caused by prostatic enlargement of the male, presence or absence of bladder neck incompetence, shape of the both ureteral orifices suggesting vesicoureteral reflux, and the degree of bladder trabeculation. Hereinafter, the term ‘baseline patient data’, ‘patient clinical data’ or ‘baseline clinical data’ collectively refers to the patient data listed above and clinical information that is obtained from non-urodynamic tests.

In general, the data obtained in the urodynamic study may include several errors or artifacts. These errors may lead to erroneous interpretation if not corrected quickly and accurately. The present invention may include artificial intelligence (AI) algorithm 192 that may be trained using a vast amount of inspection data accumulated in advance and have a function to auto-detect these errors. The AI algorithm 192 may detect some of the errors that can be avoided in advance, and accurately recognize the other errors that cannot be avoided in advance after the examination and suggest a countermeasure for the recognized errors.

In embodiments, some of the errors that the AI algorithm 192 can detect may be as follows: the errors/artifacts during uroflowmetry may include wag artifact, or artifacts due to an uneven collection of urine, abdominal straining, fecal incontinence during uroflowmetry, and artificial noise signals. In embodiments, the errors/artifacts during filling cystometry may include initial resting pressure, air bubble in the intra-abdominal pressure (Pabd) line, air bubble in the intra-vesical pressure (Pves) line, air bubble in the tubing system, repeated filling, patient position, filling rate, bowel gas, rectal contractions, synchronous change of the Pves with corresponding rectal contraction, abdominal breathing, voluntary pelvic floor muscle contraction, summation of involuntary detrusor contraction, underestimated bladder compliance, and diuresis affecting estimation of filled volume. In embodiments, the errors/artifacts during pressure-flow study include expelled Pves catheter, expelled Pves catheter, Pves catheter hole getting touched against the bladder mucosal surface, rectal contraction, flatus, urge to defecate, voluntary pelvic floor muscle contraction, involuntary detrusor contraction before permission to void, dropped Pabd at void, after-contraction, wag artifact in flow, and blockage by blood clot. In embodiments, the errors/artifacts during abdominal leak point pressure include involuntary detrusor contraction, expelled Pves catheter, dampening of Pabd, Pabd catheter slippage into the rectocele, pelvic floor laxity, and overestimation due to the catheter itself. In embodiments, the errors/artifacts during detrusor leak point pressure include involuntary detrusor contraction, and underestimation by vesicoureteral reflux. In embodiments, the errors/artifacts during pelvic muscle EMG include contact with infusate, electromagnetic interference, weak signal, urge to defecate during filling cystometry, hiccup during filling cystometry, guarding reflex affecting filling cystometry, habitual contraction of pelvic floor muscle before voiding, lower limb weakness, and lower limb spasticity. In embodiments, the errors/artifacts during urethral Pressure Profile include voluntary pelvic floor contraction, low infusate pressure, involuntary detrusor contraction, and excessively longer functional profile length. In embodiments, the errors/artifacts during fluoroscopic monitoring include overlapping symphysis pubis with bladder neck and improper timing in obtaining images.

In embodiments, the present invention may (1) determine the items of detailed sub-tests of the urodynamic study to be performed based on the baseline clinical data of the patient, (2) auto-detect errors and make a list of artifacts during performing the urodynamic study, (3) when a urodynamic study is performed, automatically select detailed findings so as to draw the results of the urodynamic study, (4) in addition to the conventional urodynamic conclusions, list other major findings obtained during the urodynamic study, and (5) if a specific nature of the lower urinary tract dysfunction is characterized, predict risk factors for the upper urinary tract dysfunction in the future. In embodiments, the detailed findings in item (3) above may include one or more information on the presence or absence of bladder neck incompetence, vesicoureteral reflux during the filling phase, trabeculation during filling phase, vesicoureteral reflux during the voiding phase, intra-prostatic reflux during the voiding phase, the behavior of bladder neck or urethral sphincter during voiding phase, and amount of the PVR at post-void stage.

FIG. 1B shows a schematic diagram of the urodynamic study 100 for acquiring urodynamic data of a lower urinary tract system according to embodiments of the present disclosure. As depicted, the system 100 may include: a urethral catheter 124 to be inserted into the bladder 108 through the urethra of the patient; a rectal balloon 122 to be inserted into the rectum 112 for intra-abdominal pressure measurements; one or more electrodes 120a and 120b attached around the anus (or urethral sphincter or pelvic floor muscles) to measure the electromyography (EMG) signal that represents the electrical activity produced by the urethral sphincter or pelvic floor muscles; an imaging device 130; a volume sensor 128 (such as weight scale, for instance) for measuring the volume (or weight) of the fluid in the container 126; and a computer 132 electrically coupled to various components of the system 100, controlling the components of the system 100 and processing the signals from the components.

In embodiments, the electrode 120a may be a needle electrode inserted into the anal sphincter or an intra-urethral electrode inserted into the urethra to measure the activity of the urethral sphincter.

FIG. 1C shows a schematic diagram of the pressure measurement system according to embodiments of the present disclosure. In embodiments, the rectal catheter 122 and urethral catheter 124 may be fluid-filled catheters, where the external pressure transducers 142 and 140 measure the pressures at the tips of the catheters 122 and 124, respectively. The pressure, Pabd, measured by the pressure transducer 142 and the pressure, Pves, measured by the pressure transducer 140 may be input to the computer 132.

As depicted in FIG. 1C, in embodiments, each of the pressure transducers 140 and 142 are coupled to stop cock. When a stopcock is open, a fluid meniscus is formed and the pressure at the corresponding pressure transducer is set to the atmospheric pressure. Then, the height of the stop cock may be set to the upper edge 153 of the pubic symphysis, and Pves and Pabd are set to zero. This process is called “zeroing” and performed in the systems in FIGS. 1B-1C.

In embodiments, the pressure sensors in the catheters 122 and 124 may communicate wireless channels, such as wi-fi or Bluetooth. In embodiments, the sensors may be MEMS devices and installed near the tip of the catheters.

In embodiments, the container 126 may collect the fluid voided by the patient and the volume sensor 128 may convert the measured weight into an electrical signal and communicate the electrical signal to the computer 132 through a wireless channel or a wire. In embodiments, the flow rate and the voided volume are interdependent since the flow rate may be calculated from the voided volume, or vice versa. In embodiments, other types of measuring devices may be used in place of the volume sensor 128. For instance, load cell (gravimetric) or rotating disc technologies may be used to measure the flow rate or voided volume. In another example, the dipstick method, which uses a capacitive technique, may be used to measure urine depth in the container 126. In yet another example, the drop spectrometry may be used to determine the flow rate by counting the rate of drops of urine leaving the meatus. In embodiments, the electrical signal from the volume sensor 128 may be transmitted through a wire or wireless communication channel and processed to determine the flow rate, flowmetric curve pattern, and the total voided volume of the patient.

In embodiments, various types of imaging device 130 may be used. For instance, the imaging device may be an x-ray fluoroscopic monitoring system to capture the image of internal organs, such as bladder 108 and urethra, of the patient. In embodiments, after contrast media-mixed fluid is infused into the bladder 108 through the urethral catheter 124, the fluoroscopic image generated by the x-ray may be captured and analyzed to diagnose the lower urinary tract dysfunction, such as urinary incontinence, anatomical abnormalities of the bladder or urethra, reflux to the kidney(s), presence of post-void residual (PVR) remaining in the bladder, etc. Hereinafter, the terms urine and fluid are used interchangeably since the physician may infuse fluid into the bladder during various tests.

In embodiments, the patient or caregiver of the patient may provide baseline clinical data 180 or clinical information to the computer 132 using a mobile device 133, such as cell phone or PDA. For instance, mobile device 133 may send the patient data to the computer 132. As discussed above, the term baseline clinical data 180 refers to, but not limited to, patient demographics (such as gender, age, height, weight, body mass index, abdominal circumference, so on), past medical history (including any neurological diseases, previous surgical history especially in brain, spinal cord, vertebrae, genitourinary system or pelvic cavity), current medication, systemic review (including erectile function, bowel function, lower urinary tract symptoms), physical examination (including motor or sensory function of the trunk, and limbs, lower abdomen or prostate), focused neurological examinations for perineum, anus and genitourinary system of the patient, parameters from voiding diary or frequency-volume chart that shows the frequency and volume, and response to the lower urinary tract symptom questionnaires. Hereinafter, the term patient data collectively refers to the patient data listed above and clinical information that is obtained from non-urodynamic tests.

In embodiments, various types of sensors may be used in place of the sensors in the system 100. For instance, a laser may be used to measure the flow rate in place of the volume sensor 128. In another example, the pressure inside the bladder may be directly or indirectly measured by the near-infrared spectroscopy (NIRS) technique applied to the patient's abdominal skin. Volume sensor 128 may be replaced by other types of sensors, such as a laser.

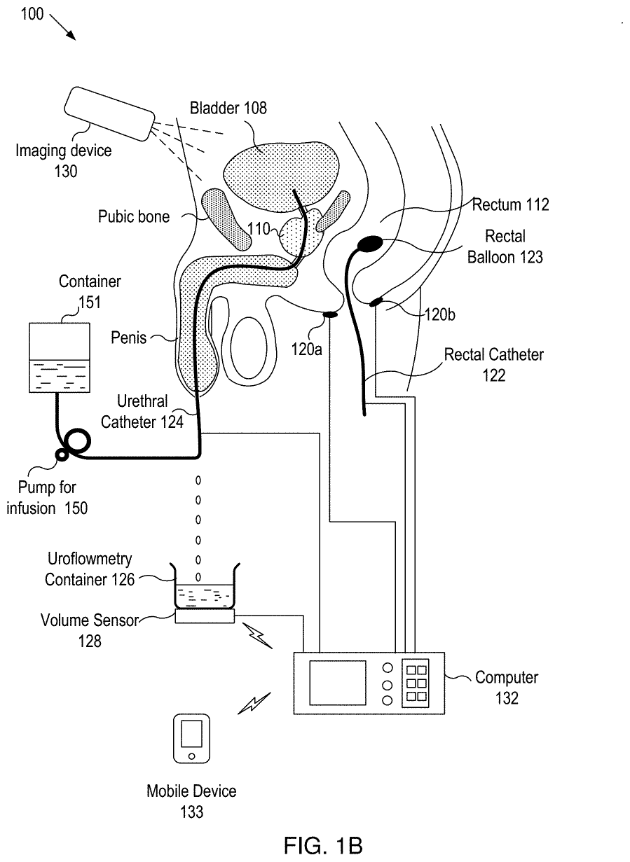

To diagnose the patient's lower urinary tract dysfunction, the physician may perform various tests for lower urinary tract dysfunction. Hereinafter, the term urodynamic test collectively refers to tests performed to characterize lower urinary tract dysfunction of a patient, such as, uroflowmetry, cystometry, urethral pressure measurement and leak point pressure measurements. In embodiments, the physician may perform uroflowmetry with the patient placed in the standing or sitting position to measure the total amount of voided volume as well as the flow rate. FIGS. 2A and 2B show exemplary data from uroflowmetry for two different patients, respectively, according to embodiments of the present disclosure. As depicted in FIG. 2A, a plot 210 may represent the volume of urine collected by the container 126 as a function of time. In embodiments, the plot 210 may reach a plateau, which may represent the total volume of urine voided by a first patient with a normal voiding function. In embodiments, a plot 220 may represent the flow rate of the first patient. In embodiments, the plots 210 and 220 may be obtained by processing the signal from the volume sensor 128.

FIG. 2B shows data from a uroflowmetry, more specifically, a flow-EMG study, where the data includes a plot 230 of EMG signal from the EMG sensor 120a (or 120b), a plot 240 representing the flow rate, and a plot 250 (volume micturition, Vmic) representing the amount of urine voided by a second patient with bladder outflow obstruction such as benign prostatic hyperplasia. In embodiments, the plots 240 and 250 may be obtained by processing the signal from the volume sensor 128. In embodiments, the flow-EMG study may be performed to determine whether the voiding pattern of the patient is synergic with the urethral sphincter activity during voiding.

In embodiments, the plot 230 of EMG signal may be used to determine the urethral sphincter activity. Normally, the bladder function should be in accordance (synergic) with urethral sphincter activity. Urethral sphincter needs to be relaxed (decrease in EMG activity) when the bladder contracts (urine flow appears) to empty the bladder, vice versa. Typically, a healthy person may show the normal active activity of urethral sphincter during the storage phase, drop in the activity of urethral sphincter right before the voiding starts and show normalization of the urethral sphincter activity after the voiding is completed. In contrast, a patient with fixed sphincter activity due to relevant neurological disorders may not be able to automatically relax urethral sphincter (therefore, no decrease in EMG activity) during voiding. Similarly, an increase in the activity of urethral sphincter during voiding, or detrusor-sphincter dyssynergia, may be observed in a patient with relevant neurological disorders, such as certain spinal cord injury or spinal cord diseases. These abnormalities of urethral sphincter activity may be determined by analyzing the plot 230.

As shown in the plot 220, the flow rate may have a peak flow rate, Qmax, 222 and the voiding time 224 may represent the time taken by the patient to void. In contrast, the plot 240 may have multiple local maximum points 242, 244 and 246. In embodiments, the plot 250 may reach a plateau, which represents the total amount of urine voided by the second patient.

Also, in embodiments, the hesitancy time 225 (shown in plot 250) may represent the time interval between the starting point of the test and the starting point of voiding.

In embodiments, after the urodynamic tests, the physician may measure the amount of PVR fluid remaining in the bladder. In embodiments, to measure the amount of PVR quantitatively, the physician may use: (1) the urethral catheter 124 to drain the urine from the bladder; (2) portable ultrasound bladder scanner; or (3) a conventional ultrasound device. Alternatively, the physician may use the fluoroscopic X-ray 130 to determine the PVR volume qualitatively.

In embodiments, several features in the plots 210, 220, 230, 240 and 250 may be used as indicators to obtain urodynamic conclusion and/or identify lower urinary tract dysfunction of the patient. In embodiments, the features may include Qmax 222 (or 242), voiding time 224, hesitancy time 225, flowmetric curve pattern (such as the number of peaks 242-246 in the uroflowmetry plot 220 or 240), the total amount of voided volume and the amount of PVR. In embodiments, the flowmetric curve pattern (220 or 240) may also be classified, such as normal bell-shaped pattern, tower-shaped superflow pattern, compressive pattern, plateau-shaped constrictive pattern, interrupted-shaped pattern, intermittent straining flow pattern, staccato-shaped pattern, so on.

For instance, it may be concluded that the second patient has intermittency since the flowmetric curve 240 has interrupted-shaped. In another example, in a case where the voiding time 224 is within a normal range, it may be suggested that the detrusor function of the first patient is likely to be normal. In yet another example, in a case where the Qmax 220 is too high (or too low) and the voiding time 224 is too narrow (or too wide), it may be suggested that the patient has the superflow (or obstructive flow) pattern.

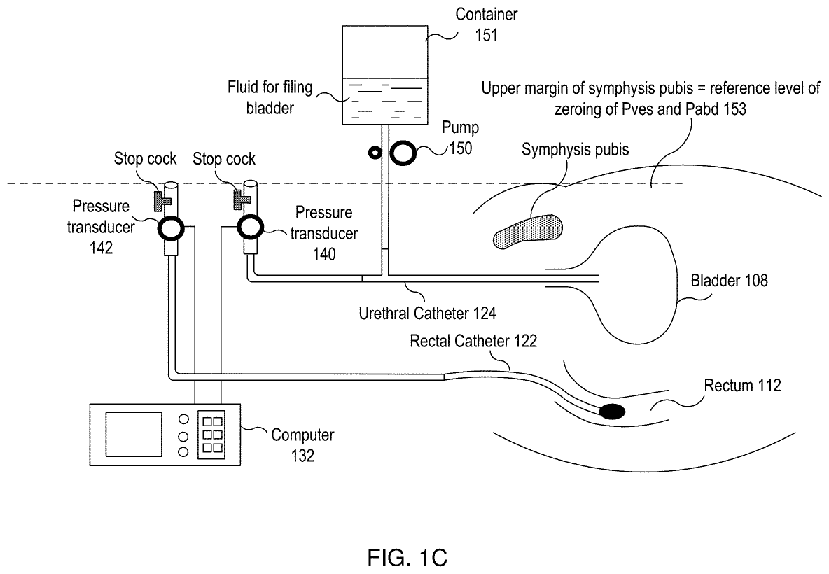

The bladder 108 has detrusor muscle that relaxes and is distended to allow the bladder to store urine, and contracts during voiding to empty urine from the bladder. In embodiments, the physician may perform cystometry (or equivalently cystometrogram), which is also an important part of urodynamic test used to investigate any dysfunction during storage (or filling) 315 and voiding (or emptying) 316 phases of the bladder. FIG. 3A shows an exemplary data 310 from cystometry for a patient according to the embodiment of the present disclosure. In embodiments, a plot 311 shows the detrusor pressure, Pdet 350, measured during cystometry, where the detrusor pressure (Pdet) may be the intrinsic pressure generated by the bladder pressure (intra-vesical pressure, Pves) 330 subtracted by intra-abdominal pressure (Pabd).

In embodiments, cystometry may include two phases: storage (or filling) phase 315 where the physician may fill the bladder with fluid using the urethral catheter (filling cystometry) 124; and a voiding phase 316 where the patient is permitted to empty the bladder (voiding cystometry, or equivalently pressure-flow study). In embodiments, the intra-intra-vesical pressure (Pves) 330, which is the pressure inside the bladder, may be measured by a pressure sensor 140 which is connected to the urethral catheter 124. In embodiments, the intra-abdominal pressure (Pabd) 340 may be measured by a pressure sensor 142 which is connected to the rectal catheter 122.

In embodiments, during the filling phase 315, the physician may fill the bladder 108 with fluid through the urethral catheter 124 at a constant rate. As shown in the plot 311, Pdet may remain low and stable until the patient feels a strong desire to urinate and is permitted to void at a point 312. At point 312, Pdet may increase rapidly since the detrusor muscle of the patient may contract to start to empty the urine. In embodiments, the cystometry during the voiding phase 316 may be termed as pressure-flow study (or equivalently voiding cystometry).

FIG. 3B shows an exemplary data of filling cystometry taken during the storage phase of cystometry in another patient according to embodiments of the present disclosure. As depicted, a plot 320 shows the amount of fluid filled in the bladder (i.e., infused volume, Vinf) through the urethral catheter 124, where the fluid may be filled at a constant rate during the storage phase. Plots 330, 340, and 350 show Pves, Pabd, and Pdet, respectively, and a plot 360 shows the EMG signal from the sensor 120a and 120b). (Here, the plot 350 may correspond to the storage phase 315 in FIG. 3A.) In the filling cystometry, the horizontal axis corresponds to time. Since the bladder is filled at a constant rate during the filling cystometry, the time eventually reflects the filling volume. Typically, a cough, which is the stress maneuver, may be used to induce an increase in intra-abdominal pressure. In embodiments, the physician may instruct the patient to cough a few times, where both Pves and Pabd show sharp peaks (such as 322 and 332) simultaneously at each cough with the same amplitude of pressure. Since Pdet 350 is the pressure of Pves 330 substracted by the Pabd 340, these peaks do not appear in the plot 350 of Pdet since the amplitude of the peaks of Pves 330 and Pabd 340 may be the same. In embodiments, the EMG signal 360 may show an increase of EMG activity due to reflex activation of pelvic muscle or urethral sphincter in the plots 320, 330, and 340. In embodiments, the physician may instruct the patient to cough before and after the voiding phase so as to ensure proper pressure transmission from the body to the pressure sensors (i.e., the coughing may be used for quality control of the measurement equipment). In embodiments, the physician may analyze the data from the filling cystometry to obtain important information on bladder compliance, bladder sensation, bladder capacity, detrusor function, and urethral function.

The bladder compliance is defined as the increase in Pdet per unit volume of fluid filled in the bladder. During the storage phase, bladder compliance may be classified as normal, low, or high. The bladder with normal compliance may be filled to a large volume with very little increase in Pdet 350. However, low compliance bladder results from various neurological disorders, such as spinal cord injury or spinal cord diseases which result in decreased elasticity within the bladder wall, fibrosis of the bladder wall, or both. This decreased elasticity of the bladder wall may be reflected in a loss of accommodation with a gradual pressure increase during the storage phase of cystometry. FIG. 3C shows an exemplary data 370 from cystometry according to embodiments of the present disclosure. In embodiments, the storage phase 315 of the plot 311 in FIG. 3A (or the plots 371 and 376 in FIG. 3C) may be generated using the two plots: the plot of Pdet (such as 350) and the plot of the amount of fluid filled in the bladder (such as 320) in FIG. 3B.

As shown in plot 371, Pdet of the compliant bladder 372 may not increase significantly until the patient starts to void at point 373. In comparison, Pdet of the low compliant bladder 374 may increase significantly at the early stage of filling the fluid, i.e., the slope of the curve 376 increase rapidly even when the volume of the bladder reaches low volume. In embodiments, the features in FIGS. 3A-3C may be used to identify the bladder compliance, where the features may include the slope of the curve 376.

In embodiments, bladder sensation may be recorded during the storage (filling) phase. When the bladder is being filled with fluid during the filling (or storage) phase 315, the physician may ask the patient to report the level of bladder fullness. In embodiments, based on the patient's report, the physician may record the stored fluid volumes when the patient had the first sensation of filling, first desire to void, and strong desire to void. Based on the information of the bladder sensation test, the physician may determine the patient's symptoms, such as bladder oversensitivity, reduced bladder sensation, absent bladder sensation, abnormal sensation, non-specific bladder awareness, and bladder pain. For the purpose of illustration, it is assumed that the patient with a normal bladder feels a strong desire to void (bladder sensation) when the volume of the fluid filled in the bladder reaches, for example, around 400 or 500 mL. However, the patient having a reduced bladder sensation may feel the first sensation of filling when the bladder volume reaches, for example, about 400 mL.

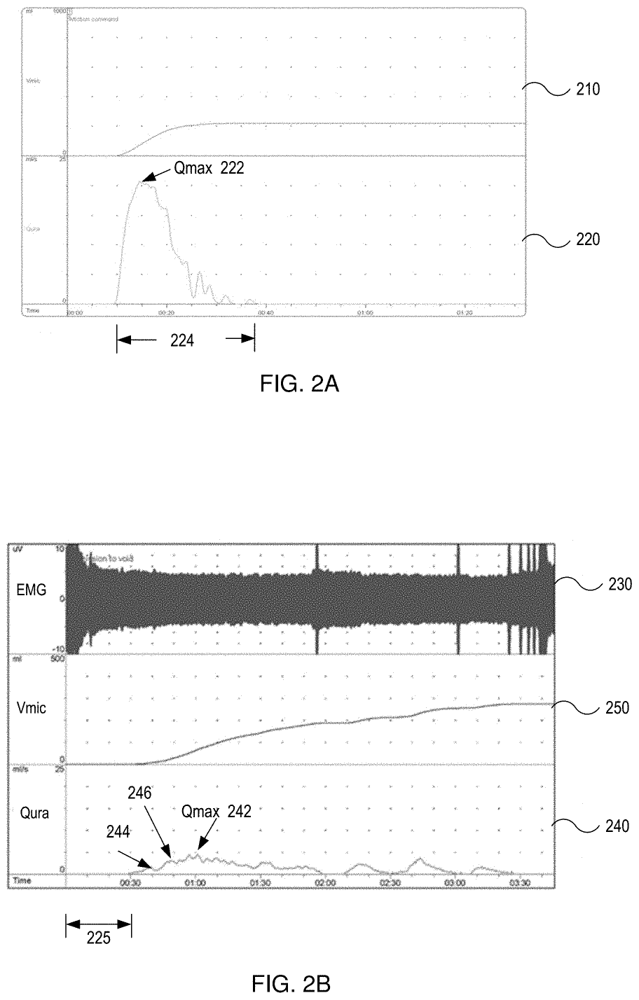

In embodiments, the physician may analyze the data from filling cystometry to characterize the detrusor activity, such as detrusor overactivity of the patient's bladder. Detrusor overactivity is defined as a urodynamic observation characterized by involuntary detrusor contractions during the storage phase that may be spontaneous or provoked. FIG. 4 shows an exemplary data 400 from filling cystometry according to embodiments of the present disclosure. As depicted, the plots 410, 420, and 430 may represent detrusor pressures (Pdet) of the bladders 412, 422, and 432, respectively. Compared to the normal bladder 412, the overactive bladder 422, 432 may have involuntary detrusor contractions 420, 430, 434, 436 during storage phase without permission to void, where the involuntary detrusor contraction may be spontaneous or provoked. In embodiments, involuntary detrusor contractions 420, 430, 434, 436 during the storage phase may indicate detrusor overactivity. Also, compared to the normal bladder 412, the bladders 422, 432 may have one or more phasic contractions 434 and 436 during the earlier stage of the storage phase.

In embodiments, the features in the plots in FIG. 4 may be used to identify urodynamic parameters and obtain urodynamic conclusion. It may be concluded that the patient has the phasic involuntary detrusor contraction 434, 436 when involuntary detrusor contractions occur during the early stage of the storage phase. It may also be concluded that the patient has terminal involuntary detrusor contraction 420, 430 when an involuntary detrusor contraction occur during the last stage of the storage phase. In both cases where the patient has involuntary detrusor contraction during the storage phase, either phasic 434, 436 or terminal 420, 430, or both, it may be concluded that the patient has detrusor overactivity.

In embodiments, the bladder capacity may be determined by the filling cystometry. Cystometric capacity is the bladder volume at the termination of the filling cystometry. In embodiments, the physician may report the reason for termination, such as, ‘strong urgency.’ A maximum cystometric capacity is the volume at which a patient with a normal bladder sensation expresses a strong desire to void.

In embodiments, the x-ray fluoroscopy image from the device 130 may be analyzed to determine incompetent bladder neck, prostatic enlargement, vesicoureteral reflux, trabeculation, bladder diverticulum, contracted bladder, and urinary leakage during the filling phase. During the voiding phase, the x-ray fluoroscopy image from device 130 may show vesicoureteral reflux at bladder contraction, intraprostatic reflux, detrusor-sphincter dyssynergia, location of the obstruction in male during the voiding phase. Also, X-ray fluoroscopic image from device 130 in the post-void period may show large amount of PVR. X-ray fluoroscopic image from device 130 may demonstrate urinary leakage during the ALPP test.

In embodiments, the urethral function may be determined during the filling cystometry. At the storage (or filling) phase 315, the urethral function may be classified into a normal or incompetent urethral closure mechanism according to the ICS standardization. A bladder having a normal urethral closure mechanism may have no leakage even when intra-abdominal pressure increases suddenly (ALPP test), although leakage could occur due to detrusor overactivity at the storage phase. A bladder may have an incompetent urethral mechanism when urinary leakage occurs in the absence of bladder contraction. Urodynamic stress urinary incontinence is defined as the involuntary leakage that occurs when intra-abdominal pressure increases in the absence of bladder contraction.

In general, the ICS does not provide quantitative criteria that specify what study to do or what cutoff values to apply to determine normal or incompetent urethral closure mechanism. Since there is no guideline as to which one of urethral pressure profile (UPP) or ALPP must be measured to determine the type of urethral closure mechanism, the physician may judge the urethral closure mechanism based on his own clinical experience. In embodiments, to characterize urethral function at storage phase 315, the physician may make a clinical judgment based on the patient's history, physical examination, and upright position ALPP along with the bladder neck state evaluated by fluoroscopy. In embodiments, the physician may check for the open bladder neck at low bladder volumes with x-ray fluoroscopy in making the judgment.

In embodiments, DLPP may be measured during the filling cystometry. DLPP is defined as “the lowest intra-vesical pressure at which leakage is noted around catheter during the bladder filling phase 315. DLPP is a parameter to detect the “high-pressure bladder” that may cause upper urinary tract deterioration in patients with neurogenic bladder. To measure DLPP, the patient is placed in a supine position and the bladder is emptied. The bladder is filled with a predetermined filling rate, such as 5-10 or 60 mL/min, where the patient needs to relax, not strain, not suppress a bladder contraction, and not inhibit leakage during the filling phase. DLPP may also be defined as the pressure at which urine leakage occurs in the absence of a detrusor contraction or abdominal pressure increase. A leak may be detected either by x-ray fluoroscopy or by a direct vision of the urine at the external urethral meatus. Typically, the parameter measured is Pves 330.

In embodiments, for a patient with neurogenic bladder, DLPP above 40 cmH2O may be considered as a high-risk factor for upper urinary tract deterioration in the future. In embodiments, for a patient with poor bladder compliance, DLPP may be used as a predictor of upper tract damage. In embodiments, the measured DLPP may be considered when the physician makes the urodynamic conclusion.

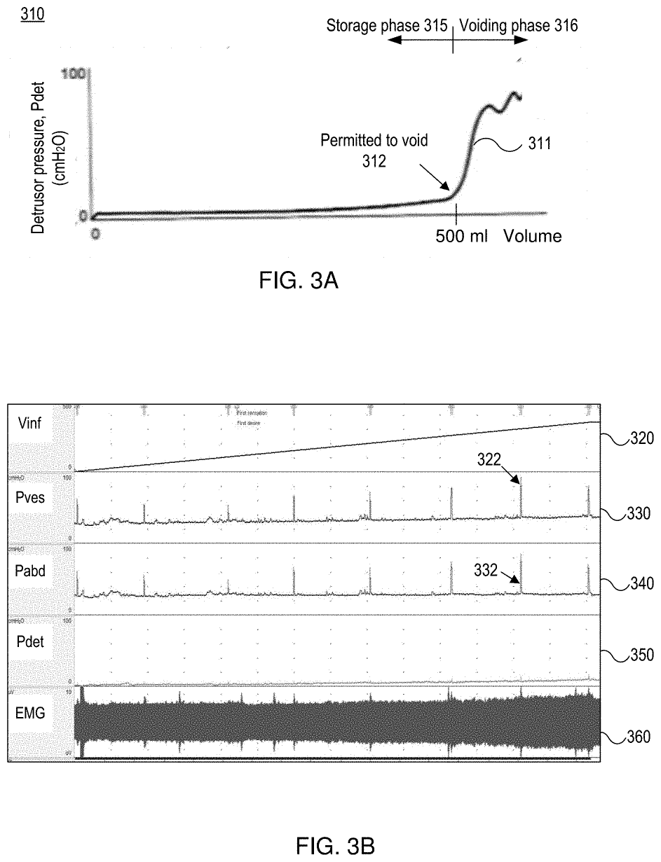

FIG. 5 shows an exemplary data taken during the voiding phase of cystometry (pressure-flow study) according to embodiments of the present disclosure. As depicted, the plots 510, 520, and 530 show Pves, Pabd, and Pdet, respectively, measured during the voiding phase of cystometry 316, and the plot 540 shows the EMG signal from the sensor 120a (or 120b). In embodiments, the EMG signal 540 may be used to detect urethral sphincter activity and determine whether the uroflow is synergic or dyssynergic with the urethral sphincter (i.e, determine detrusor-sphincter synergia or dyssynergia). As explained above in the flow-EMG study, a healthy person may show an increase in the activity of urethral sphincter as the bladder fills, drop in the activity of urethral sphincter right before the voiding starts and resume the activity of urethral sphincter after the voiding is completed. In contrast, patients with a fixed sphincter activity due to certain neurological disorders may not be able to decrease the activity of urethral sphincter during voiding. Similarly, a patient with detrusor-sphincter dyssynergia due to certain neurological disorders may increase the activity of urethral sphincter during voiding. As the EMG signal 540 may have errors and/or artifacts, the x-ray images of the bladder neck and external sphincter may be obtained right before and after voiding to determine whether the bladder neck and external sphincter are open.

In embodiments, the plot 550 shows the uroflowmetry, i.e., the flow rate of the urine, Qura, where the plot 550 may be obtained by processing the data from the volume sensor 128. In embodiments, immediately before and after the voiding phase, the physician may instruct the patient to cough 562, 564. Also, as discussed above, the physician may instruct the patient to cough before and after voiding to ensure proper pressure transmission from the body to the pressure sensors (i.e., the coughing may be used for quality control of the measurement equipment). In embodiments, the sharp peaks in the Pves 562, 564 and Pabd 566, 567 correspond to the pressure responses to the sudden increases of the whole abdominal pressure. In embodiments, the urine starts to flow at point 552 and ends flowing at point 556, and the flow rate has the maximum value at point 554. In embodiments, the points 532 and 534 correspond to the starting and end points 552 and 556 of the voiding, respectively, and Pdet at the two points correspond to the opening detrusor pressure and closing detrusor pressure, respectively. In the plot 530, PdetQmax 580 is the detrusor pressure at the point where the flow rate Qura is at its maximum during the voiding phase, PdetQmin 582 is minimum detrusor pressure during the voiding phase, opening detrusor pressure 532 is the detrusor pressure where the voiding starts, and closing detrusor pressure 534 is the detrusor pressure where the voiding ends. In embodiments, several features, which may include Qmax 554, PdetQmax 580, PdetQmin 582, the opening detrusor pressure 532, the closing detrusor pressure 534, and the total amount of voiding, may be used to identify urodynamic parameters and obtain urodynamic conclusion.

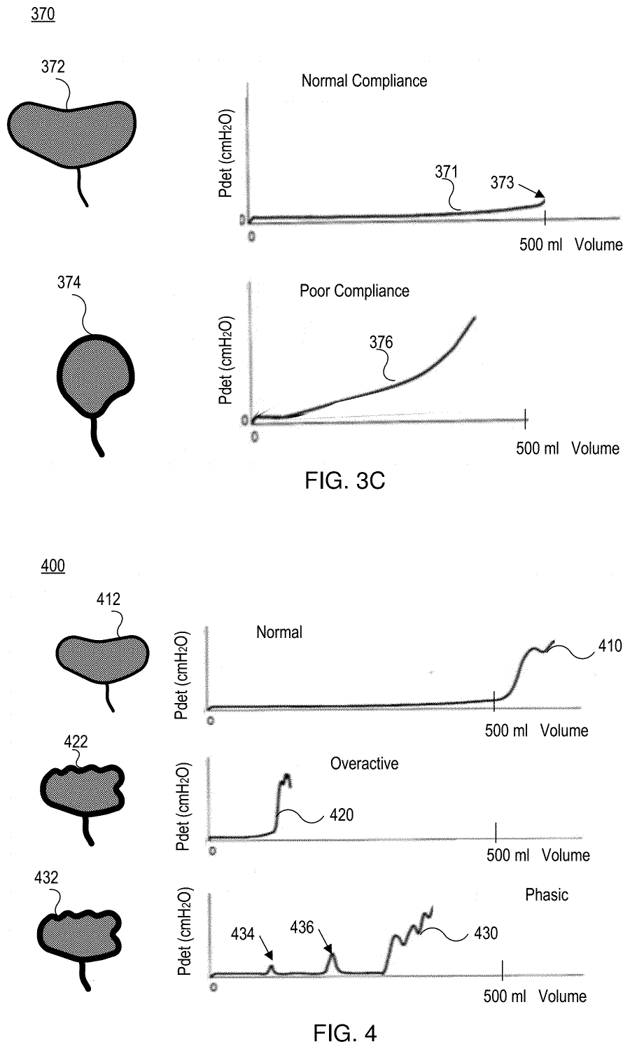

FIG. 6 shows a plot 600 of Pdet as a function of urine flow rate, Qura, according to embodiments of the present disclosure. In embodiments, the plots 530 and 550 may be used to generate the plot 610. As depicted, the entire domain of the plot 600 may be divided into three regions: obstructed, unobstructed, and equivocal regions. Since the plot 610 is in the unobstructed region, it may be concluded that bladder is not obstructed. In contrast, for a patient having benign prostatic hyperplasia that blocks the flow of urine through the urethra, the plot of Pdet vs. Qura may have a pattern similar to the plot 620. In such a case, it may be concluded that the patient shows the mechanical bladder outflow obstruction. Thus, in embodiments, the pattern of the plot of Pdet versus flow rate may be used as a feature to obtain the urodynamic conclusion. To numerically quantify the bladder outflow obstruction, a specific formula may be used to draw bladder outlet obstruction index (BOO index);

BOO index=PdetQmax−2*Qmax,

where the male bladder outflow may be classified into obstructed, equivocal, and unobstructed according to their BOO index: BOO index>40 (obstructed); BOO index 20-40 (equivocal); and BOO index<20 (unobstructed).

In embodiments, the x-ray image from the device 130 may be used during the pressure-flow study to determine various parameters, such as vesicoureteral reflux at bladder contraction, intra-prostatic reflux, detrusor-sphincter dyssynergia, and incompetent bladder neck.

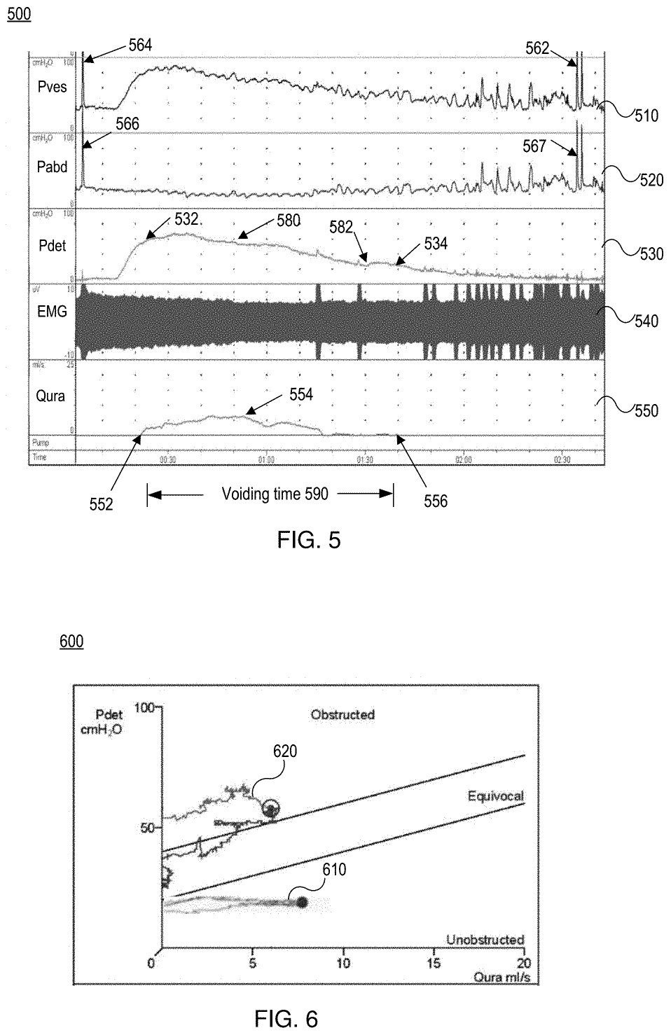

In FIG. 5, the voiding time 590 represents the time span to empty the bladder. Detrusor underactivity is defined as a contraction of reduced strength and/or duration, resulting in prolonged bladder emptying and/or a failure to achieve complete bladder emptying within a normal time span. FIG. 7 shows an exemplary data 700 from cystometry (where that of the voiding phase corresponds to the pressure-flowmetry) according to embodiments of the present disclosure. As depicted, a plot 710 may represent Pdet of a normal bladder 712, while a plot 720 may represent Pdet of an underactive detrusor 722. During the voiding phase 702, the underactive bladder (i.e., a bladder having underactive detrusor) 722 may contract with reduced strength, Pdet, resulting in a failure to achieve complete bladder emptying. To numerically quantify the degree of bladder contractility, the following formula may be used to draw bladder contractility index (BCI);

BCI=PdetQmax+5*Qmax.

In embodiments, the physician may conclude that a bladder has weak contractility when BCI is less than 100; a normal contractibility when BCI ranges 100-150; and a strong contractility when BCI is larger than 150. In embodiments, another parameter which may be used to represent the degree of bladder emptying in the underactive bladder is bladder voiding efficiency (BVE), which is defined as a percentage according to the following equation;

BVE=(voided volume/total bladder capacity)×100.

For instance, if a patient empties 150 mL leaving a PVR volume of 350 mL (total bladder capacity 500 mL), then the patient has a BVE of 30%. In embodiments, the features, which includes the maximum Pdet, BCI, and BVE during the voiding phase and the presence of PVR volume, may be used to identify urodynamic parameters and obtain urodynamic conclusion.

In this case, the physician may measure the PVR volume in a qualitative manner using the fluoroscopic imaging device 130 after completion of voiding. For instance, the physician may infuse contrast media inside the bladder through the urethral catheter 124 and get a fluoroscopic image generated by the x-ray fluoroscopic imaging device 130 to estimate PVR volume using the image.

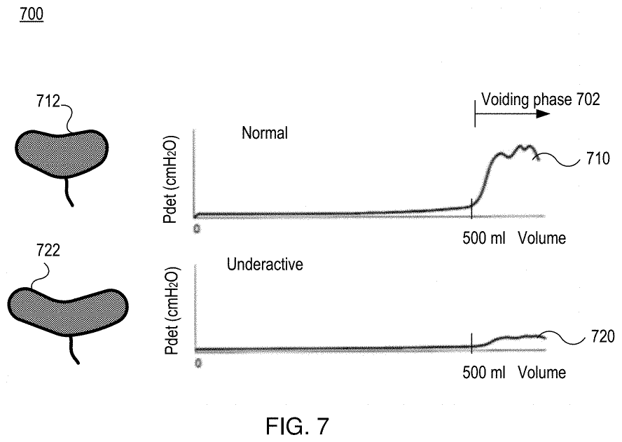

The external urethral sphincter muscle (hereinafter, shortly, sphincter) is located right below the prostate 110 and control the opening/closure of the flow of urine from the bladder 108. In embodiments, the sphincteric competence may be estimated, though not solely, by analyzing the urethral pressure profiles. FIG. 8 shows an exemplary data 800 taken during a urethral pressure profile measurement according to embodiments of the present disclosure. During the urethral measurement, the physician may insert the urethral catheter 124 into the bladder 108 and continuously infuse fluid through the second hole of the catheter into the bladder at a pre-defined constant rate while the urethral catheter 124 is slowly being withdrawn along the urethra at a pre-defined constant withdrawal rate. In embodiments, the urethral catheter 124 may be modified to have two holes that are located at the distal end portion of the catheter and separated by about 6 cm from each other along the longitudinal direction of the catheter. In embodiments, the intra-vesical pressure, Pves 820, may be continuously measured by a pressure sensor located near the first hole of the catheter and the urethral pressure, Pura 850, may be continuously measured by a pressure sensor located near the second hole of the catheter while the catheter may be pulled out at a constant rate along the urethra and fluid is infused through the second hole.

In FIG. 8, the plot 820 represents the intra-vesical pressure (Pves) measured through the hole located at the tip of the catheter. The plot 850 represents the urethral pressure measured through the second hole (Pura) at the tip of the catheter, and the plot 860 represents the urethral closure pressure which corresponds to the Pura 850 subtracted by the Pves 820. In embodiments, the maximum urethral closure pressure 866 may be obtained by subtracting Pves 820 from the maximum urethral pressure 864. By way of example, the maximum urethral closure pressure 866 is 68 cmH2O and the maximum urethral pressure 864 is 72 cmH2O. In FIG. 8, the pressure 868 may represent the resting bladder pressure and the width 862 may represent the (functional profile) length of a portion of the urethral where the urethral pressure is higher than Pves. By way of example, as shown in FIG. 8, the resting bladder pressure may be 6 cmH2O and the functional profile length may be 74 mm. The maximum urethral pressure, maximum urethral closure pressure, and functional profile length may be used to identify parameters that represent the static functional aspects of the urethra.

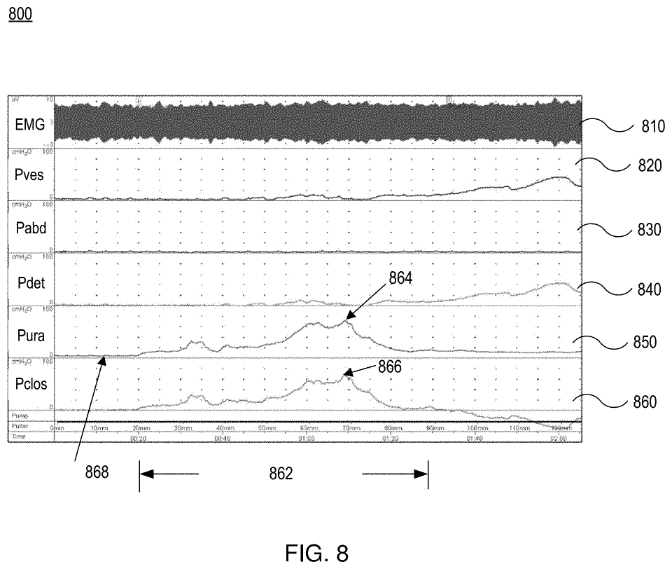

In embodiments, the ALPP may be used as a tool for determining the severity of stress urinary incontinence and the presence of intrinsic sphincter deficiency. Practically, the physician may measure two types of ALPP: Valsalva leak point pressure (VLPP) and cough-induced leak point pressure (CLPP). VLPP refers to the value of the intra-vesical pressure, Pves 910, which exceeds the continence mechanism and results in a leakage of urine in the absence of a detrusor contraction, while CLPP refers to the value of the intra-vesical pressure that is induced by a cough and results in a leakage of urine. FIG. 9 shows an exemplary data 900 from an ALPP measurement according to embodiments of the present disclosure. As depicted, the plots 910, 920, 930 and 940 show Pves, Pabd, Pdet, and EMG signal, respectively, where the plots in FIG. 9 may be obtained in a similar manner as the plots in FIG. 3B.

In embodiments, during the ALPP measurement, the physician may instruct the patient to cough several times, as indicated by the sharp peak points 902, while the fluoroscopic image of the bladder, which may be generated by the imaging device 130, may be analyzed to check the cough-induced urinary incontinence, i.e., involuntary leakage of urine. Using plot 910, the physician may determine CLPP 912, which corresponds to a minimum cough-induced abdominal pressure that caused the patient to urinary leak. Similarly, the physician may determine VLPP 922, which corresponds to a minimum abdominal straining pressure that caused the patient to leak urine. In embodiments, the urinary leakage may be important in determining the point in time when ALPP occurs. In embodiments, the measured VLPP and CLPP may be used to identify urodynamic parameters and obtain urodynamic conclusion for determining the severity of stress urinary incontinence and intrinsic sphincter deficiency.

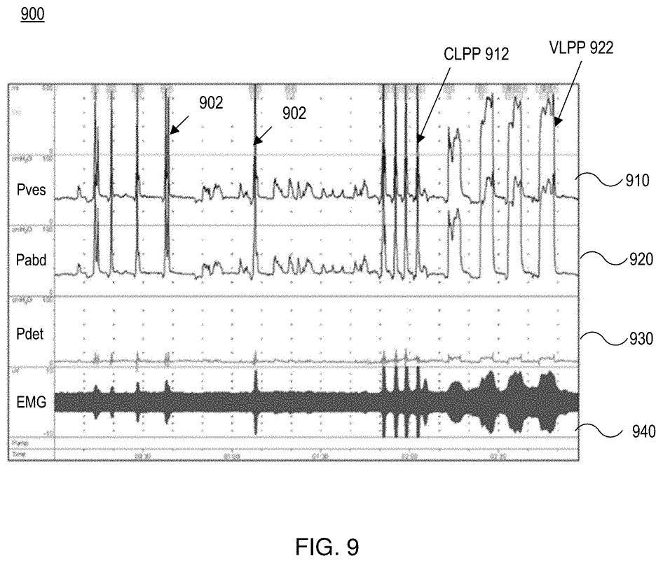

FIG. 10 shows a table 1000 of the urodynamic conclusion according to the ICS classification. As depicted, each item in Table 1000 may a clinical or urodynamic parameter (or shortly parameter) that correspond to one or more the lower urinary tract dysfunctions. For instance, based on urodynamic features in plot 420, the physician may conclude that the bladder of the patient is overactive (“detrusor overactivity”). The physician may identify other urodynamic parameters based on features extracted from the other plots in FIGS. 2A-9, and may conclude the lower urinary tract dysfunction associated with the identified parameters.

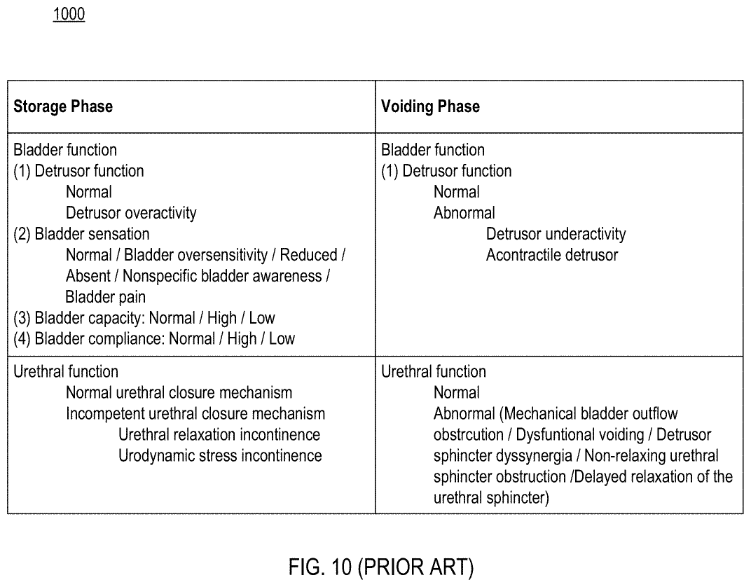

FIG. 11 shows a schematic diagram of a system 1100 for diagnosing lower urinary tract dysfunction according to embodiments of the present disclosure. As depicted, the system 1100 may include: a data acquisition station 1102 for acquiring data from the baseline clinical data 180 or urodynamic study results 186; a diagnostic system 1104 that includes a database 1106 for storing data and a diagnostic engine 1108 for diagnosing the lower urinary tract dysfunction of the patient based on the data; and a network 1112 through which the data acquisition station 1102 and diagnostic system 1104 may communicate with each other. (The urodynamic study results 186″ collectively refers to the tests described in conjunction with FIGS. 1B-9.) It is noted that the system 1100 may include other devices connected to the network 1112. For instance, the diagnostic system 1104 may report the diagnosed disorder to a physician's computer (not shown in FIG. 11) that is connected to the network 1112. In another example, a database 1120 may be located remotely from and communicatively coupled to the data acquisition station 1102 and diagnostic system 1104.

In embodiments, each of the two components 1102 and 1104 may be a computer. Alternatively, one or more of the elements in the component 1102 (and/or 1104) may be implemented as a separate computing facility. For instance, the database 1106 may be physically separated from and communicatively coupled to the diagnostic engine 1108.

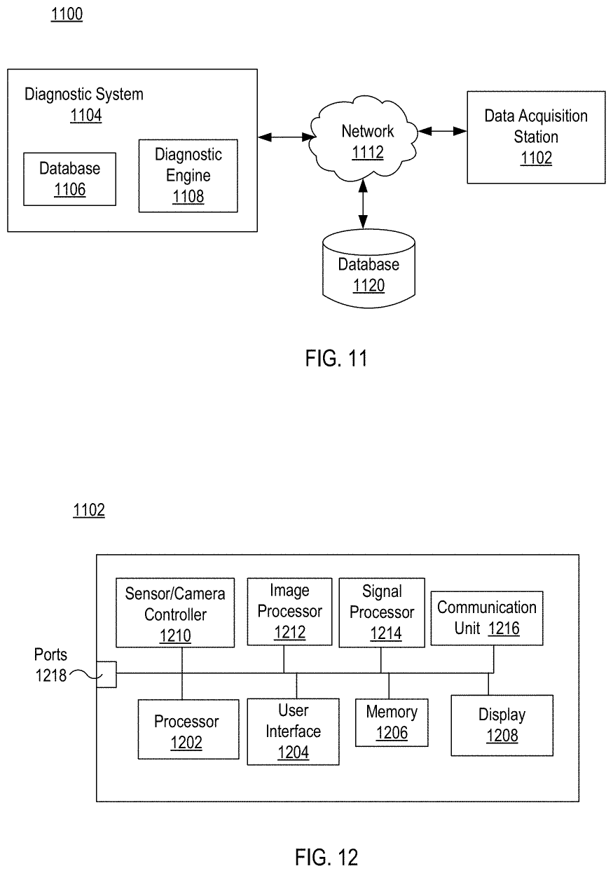

FIG. 12 shows a schematic diagram of the data acquisition station 1102 according to embodiments of the present disclosure. In embodiments, the data acquisition station 1102 may be similar to computer 132 in FIG. 1B. As depicted, the data acquisition station 1102 may include: a processor 1202, such as a microprocessor, for operating/orchestrating other components of the data acquisition station; a user interface 1204 for accepting input control signals and data from the user of the station; a memory 1206 for storing data; a display 1208 for displaying various images; a sensor/camera controller 1210 for controlling the various sensors/electrodes and imaging device 130 in FIGS. 1B and 1C to acquire data therefrom; an image processor 1212 for processing and analyzing the images, such as x-ray and ultrasound images, received from the imaging device 130; a signal processor 1214 for processing signals acquired by the sensor/camera controller 1210; a communication unit 1216 for communicating data with various external devices, such as sensors and imaging devices, as well as nodes/stations coupled to the network 1112; and one or more ports 1218 for accepting various terminals, such as power cable, USB, so on.

In embodiments, as discussed above, the sensor/camera controller 1210 may control the imaging device 130 to get an image of internal organs, such as bladder 108, of the patient, where the imaging device 130 may be an x-ray fluoroscopic monitoring system. Also, the sensor/image controller 1210 may control the sensors/electrodes to get signals shown in FIGS. 2A-9.

In embodiments, the user interface 1204 may include a keyboard and/or mouse for entering patient data. In embodiments, the display 1208 may be a touchscreen that allows the user to interact with the data acquisition station 1102.

In embodiments, the ports 1218 may accept various terminals so that the sensors in FIGS. 1A-1B may communicate signals to the data acquisition station 1102. In embodiments, the port 1218 may include a wireless communication connection that communicates wireless signals with the sensors in FIGS. 1B-1C.

Referring back to FIG. 11, in embodiments, the diagnostic engine 1108 may receive data from the data acquisition station 1102 and analyze the received data to diagnose the patient's lower urinary tract dysfunction, where the received data may include data from the urodynamic test and the patient data. In embodiments, the diagnostic engine 1108 may be software, a hardware device, or a combination thereof. In embodiments, the diagnostic engine 1108 may include an artificial intelligence (AI) algorithm. In embodiments, the diagnostic engine 1108 may extract features from the received data, identify the outcome of the baseline clinical data and/or urodynamic parameters and characterize the patient's lower urinary tract dysfunction from the identified outcome of the baseline clinical data or urodynamic parameters. In embodiments, the diagnosed lower urinary tract dysfunction may include one or more of the items in Table 1000.

In embodiments, the diagnostic engine 1108 may extract features from the data shown in FIGS. 2A-9 that the physician would extract from the same data, i.e., the diagnostic engine 1103, more specifically the AI algorithm, may replace the physician to extract the features described in conjunction with FIGS. 2A-9. For instance, In another example, as discussed in FIG. 5, the diagnostic engine 1108 may extract features of the data taken during the voiding phase of cystometry and include: the maximum flow rate, Qmax, detrusor pressure at Qmax, detrusor-sphincter synergia (or dyssynergia), opening detrusor pressure, closing detrusor pressure, minimum detrusor pressure during the voiding phase. In another example, as discussed in FIGS. 6 and 7, the features may be obtained from various features of the data taken during the cystometry and include the bladder outlet obstruction index (BOOI), bladder contractility index (BCI) and bladder voiding efficiency (BVC). In another example, as discussed in FIG. 8, the features may be obtained from various features of the data taken during the urethral pressure profile measurement and include: the maximum urethral pressure, maximum urethral closure pressure, total profile length and functional profile length. In another example, as discussed in FIG. 9, the features may be obtained from various features of the data taken during the ALPP measurement (including VLPP and CLPP).

In embodiments, the diagnostic engine 1108 may extract parameters from the baseline clinical data 180 that is generated from clinical evaluation: major lower urinary tract symptoms, medical history of the patient (including past medical history, previous surgical history, and/or medication history), physical examination (including general neurological examination, such as sensation of limbs; focused neurological examination), questionnaire, voiding diary (or frequency-volume chart), post-void residual urine volume, status of kidney (for example, CT or ultrasound imaging), and cystourethroscopy.

In embodiments, the diagnostic engine 1108 may be trained by a supervised learning process. During the training phase, the diagnostic engine 1108 may use training data, where each of the training data may include a pair of an input object and the desired output object. In embodiments, the input object may include one or more of the data from the baseline clinical data 180 and urodynamic study results 186, such as plots/data shown in FIGS. 2A-9, where the desired output object may be associated with the input object and include the lower urinary tract dysfunction of the patient listed in Table 1000. In embodiments, an experienced physician may analyze a large number of input objects and diagnose the patient's lower urinary tract dysfunction for each input object to prepare the desired output object.

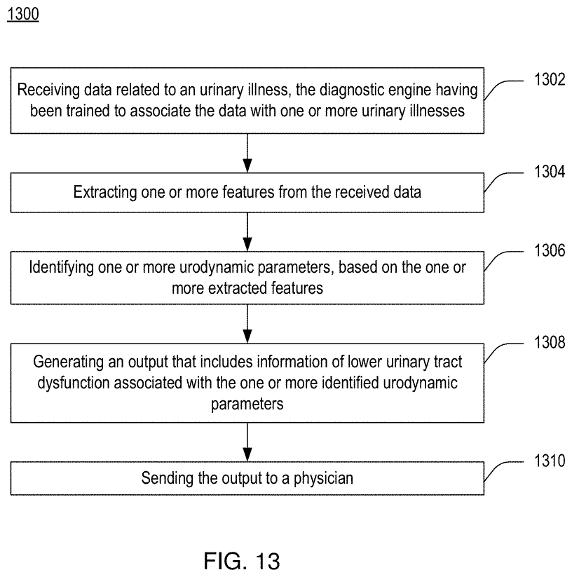

In embodiments, the trained diagnosis engine 1108 may be used for receiving a new input object and diagnosing the patient's lower urinary tract dysfunction. FIG. 13 shows a flowchart 1300 illustrating an exemplary process for operating a trained diagnostic engine 1108 to diagnose a patient's lower urinary tract dysfunction according to embodiments of the present disclosure.

At step 1302, the diagnostic engine 1108 may receive data related to a lower urinary tract dysfunction. In embodiments, the diagnostic engine 1108 may have been trained to associate the data with one or more lower urinary tract dysfunction. In embodiments, the received data may include data from the urodynamic test results 186 (as discussed in conjunction with FIGS. 2A-9) and the baseline clinical data 180 that is generated from clinical evaluation. Then, at steps 1304, the diagnostic engine 1108 may extract one or more features from the received data. Next, at step 1306, the diagnostic engine 1108 may identify one or more outcome of the baseline clinical data 180 and/or urodynamic test results 186 (or shortly, urodynamic parameters) of the patient, based on the extracted features.

In embodiments, as described in conjunction with FIGS. 2A-2B, the data received by the diagnostic engine 1108 may include plots 240 (or 220), 230 and 250 (or 210) in FIGS. 2A and 2B, where these plots may be obtained from uroflowmetry and represent flow rate, EMG signal and the voided volume, respectively. In embodiments, at step 1304, the diagnostic engine 1108 may extract features from the input data, where the features may include the flowmetric curve pattern of the plot 240 of voiding, the maximum flow rate 222 (or 242), the voiding time 224, hesitancy time, the total voided volume, and the PVR volume. In embodiments, at step 1306, the diagnostic engine 1108 may use the extracted features to identify urodynamic parameters (or shortly parameters) associated with the patient's lower urinary tract dysfunction. For instance, the diagnostic engine 1108 may identify the intermittent flow pattern when the plot 240 of the flow rate has multiple peaks. In another example, in a case where the voiding time 224 is within a normal range if the voided volume is adequate (preferably above 150 mL), the diagnostic engine 1108 may conclude that the urinary flow of the patient is normal. In yet another example, in a case where the Qmax 220 is too high (or too low) and the voiding time 224 is too narrow (or too wide), the diagnostic engine 1108 may identify superflow (or obstructive flow) pattern.

In embodiments, the data received by the diagnostic engine 1108 may include one or more of the plots 320, 330, 340, 350, and 360 in FIG. 3B, where these plots may be taken during the storage phase of cystometry and represent the bladder volume, Pves, Pabd, Pdet, and EMG signal, respectively. In the filling cystometry, the horizontal axis corresponds to time. Since the bladder is filled at a constant rate during the filling cystometry, the time eventually reflects the filling volume. In embodiments, the diagnostic engine 1108 may use these plots 311 (371 or 376). In embodiments, at steps 1304 and 1306, the diagnostic engine 1108 may extract features from the plots in 3A-3C to identify bladder compliance, where the features may include the slope of the curve 376 during the storage phase and the bladder volumes when the patient has the first sensation of filling, first desire to void, and strong desire to void (bladder sensation). For instance, if the slope of the curve 376 increases rapidly at the earlier stage of the storage phase, the diagnostic engine 1108 may identify low bladder compliance. In another example, if the patient has a strong desire to void when the bladder volume has not reached the expected average normal capacity, the diagnostic engine 1108 may identify low bladder capacity.

In embodiments, the data received by the diagnostic engine 1108 may include plots 330 and 350 in FIG. 3B, where these plots may represent Pves, Pdet, respectively. In embodiments, at steps 1304 and 1306, the diagnostic engine 1108 may extract features that include DLPP, and based on the extracted features, the diagnostic engine 1108 may identify urodynamic parameters for risk factors for the upper urinary tract deterioration in the future.

In embodiments, the diagnostic engine 1108 may generate the plots 410, 420 and 430 in FIG. 4 using the data 320, 330, 340 and 350 received by the diagnostic engine 1108, where the data may be taken during the storage phase of cystometry. In embodiments, at steps 1304 and 1306, the diagnostic engine 1108 may extract features, such as the number of involuntary contractions during the storage phase, and identify phasic contraction when multiple involuntary detrusor contractions occur during the storage phase. In embodiments, the diagnostic engine 1108 may extract another feature, such as type of involuntary detrusor contraction during the storage phase and identify parameters, such as (1) detrusor overactivity when the involuntary detrusor contraction occurs during the earlier stage of the storage phase and (2) terminal detrusor overactivity when the bladder contracts without permission to void near or at the maximum cystometric capacity. The diagnostic engine 1108 may also receive x-ray images to extract features, such as bladder neck feature (open or closed) and leakage detection from the x-ray image.

In embodiments, the data received by the diagnostic engine 1108 may include the plots 510, 520, 530, 540 and 550 in FIG. 5, where these plots represent Pves, Pabd, Pdet, EMG, and flow rate, respectively. In embodiments, the EMG signal 540 or x-ray fluoroscopy image during the voiding phase may be used to determine detrusor-sphincter synnergia (or dyssynergia). In embodiments, the diagnostic engine 1108 may generate plots 610 and 710 (or 720) in FIGS. 6 and 7 using the input data.

In embodiments, at steps 1304 and 1306, the diagnostic engine 1108 may extract features from the input data and identify various results from the features, where the features may include Qmax 554, the opening detrusor pressure 532, the closing detrusor pressure 534, the total volume of voided fluid, the pattern (610 or 620) of the plot of Pdet versus flow rate, the maximum Pdet at maximal flow rate (PdetQmax) during the voiding phase and the volume of PVR. For instance, as shown in plot 720, the diagnostic engine 1108 may identify detrusor underactivity when the maximum Pdet is low during the voiding phase. In another example, when plot 620 of Pdet versus flow rate is in the obstructed region, the diagnostic engine 1108 may identify bladder outflow obstruction.

In embodiments, the data input to the diagnostic engine 1108 may include the plots 810, 820, 830, 840, 850 and 860 in FIG. 8, where these plots may be taken during a urethral pressure profile measurement and represent EMG signal, intra-vesical pressure, abdominal pressure, detrusor pressure, urethral pressure near the tip of a catheter, and urethral closure pressure, respectively. In embodiments, the diagnostic engine 1108 may generate the plot of urethral closure pressure 860 using the plots 820 and 850. In embodiments, at step 1304, the diagnostic engine 1108 may extract a feature(s) from the plots in FIG. 8, where the feature may include the maximum urethral pressure 864, maximum urethral closure pressure, and functional profile length. In embodiments, at step 1306, the diagnostic engine may identify parameters, such as indirect evidence of sphincter incompetence when the maximum urethral closure pressure 864 is low.

In FIG. 8, the plots 810, 820, 830, and 840 are similar to their counterpart plots in FIG. 3B, i.e., the plots 810, 820, 830, and 840 represent the EMG signal, vesical pressure, abdominal pressure and detrusor pressure, respectively. The plot 850 represents the pressure measured by the pressure sensor 182 at the tip of the catheter 124, and the plot 860 represents the pressure difference between the pressure 850 measured by the sensor 182 and the vesical pressure 820, where the pressure difference is termed as urethral pressure.

In embodiments, the data received by the diagnostic engine 1108 may include plots 910, 920, 930 and 940 in FIG. 9, where these plots may represent Pves, Pabd, Pdet, and EMG signal, respectively. In embodiments, at steps 1304 and 1306, the diagnostic engine 1108 may extract features that include VLPP and CLPP, and based on the extracted features, the diagnostic engine 1108 may identify parameters, such as the severity of stress urinary incontinence and intrinsic sphincter deficiency.

At step 1308, the diagnostic engine 1108 may generate output that includes information of one or more lower urinary tract dysfunction associated with the identified urodynamic parameters. In embodiments, the identified urodynamic parameters may include one or more items in Table 1000. It is noted that the patient's lower urinary tract dysfunction may be associated with one or more abnormal baseline clinical data 180 or urodynamic parameters and that different lower urinary tract dysfunctions may have common parameters. At step 1310, the output may be sent to a physician's computer so as to assist the physician to diagnose and treat the patient's lower urinary tract dysfunctions.

It is noted that the physicians may not be able to perform some of the measurements described in conjunction with FIGS. 2A-9. For instance, a patient who underwent rectal cancer surgery and does not have the anus may have bowel movements through a stoma formed in the anterior abdomen. In such a case, in embodiments, the rectal catheter 122 may be inserted through the abdominal stoma to measure intra-abdominal pressure. Similarly, a patient who does not have the urethra may have a suprapubic cystostomy catheter indwelled in the anterior abdomen. In such a case, in embodiments, the suprapubic cystostomy catheter may serve urethral catheter 124 to measure the intra-vesical pressure.

In embodiments, the data in FIGS. 2A-9 may be obtained in the standard urodynamic study, where the patient is in a fixed position during the study. Alternatively, the same data may be obtained in the ambulatory urodynamic study, where the patient may ambulate with urodynamic pressure transducers and signal recording devices in place during the study.

In embodiments, one or more computing systems may be configured to perform one or more of the methods, functions, and/or operations presented herein. Systems that implement at least one or more of the methods, functions, and/or operations described herein may comprise an application or applications operating on at least one computing system. The computing system may comprise one or more computers and one or more databases. The computer system may be a single system, a distributed system, a cloud-based computer system, or a combination thereof.

It shall be noted that the present disclosure may be implemented in any instruction-execution/computing device or system capable of processing data, including, without limitation laptop computers, desktop computers, and servers. The present invention may also be implemented in other computing devices and systems. Furthermore, aspects of the present invention may be implemented in a wide variety of ways including software (including firmware), hardware, or combinations thereof. For example, the functions to practice various aspects of the present invention may be performed by components that are implemented in a wide variety of ways including discrete logic components, one or more application-specific integrated circuits (ASICs), and/or program-controlled processors. It shall be noted that the manner in which these items are implemented is not critical to the present invention.

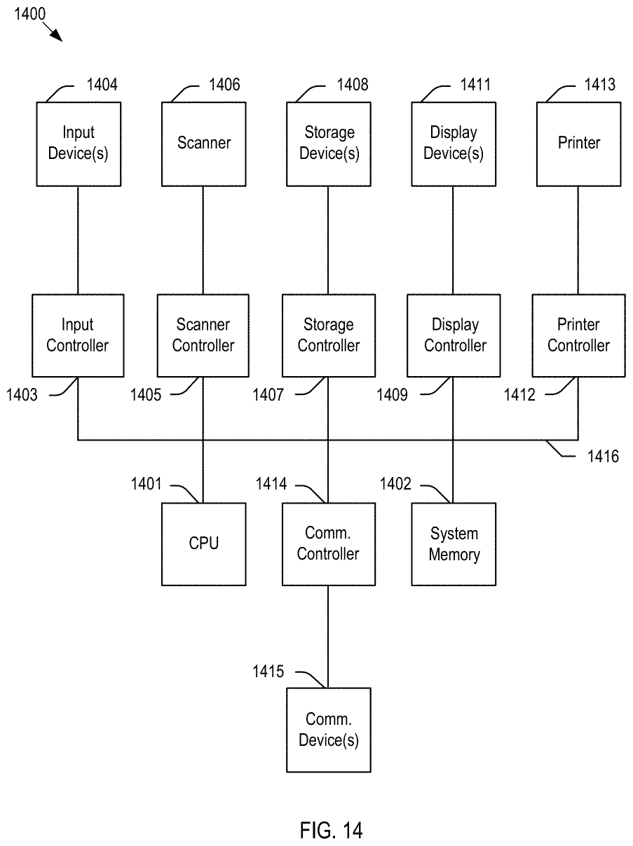

Having described the details of the invention, an exemplary system 1400, which may be used to implement one or more aspects of the present invention, will now be described with reference to FIG. 14. The computing devices described in conjunction with FIGS. 1A-12, may include one or more components in the system 1400. As illustrated in FIG. 14, system 1400 includes a central processing unit (CPU) 1401 that provides computing resources and controls the computer. CPU 1401 may be implemented with a microprocessor or the like, and may also include a graphics processor and/or a floating-point coprocessor for mathematical computations. System 1400 may also include a system memory 1402, which may be in the form of random-access memory (RAM) and read-only memory (ROM).

A number of controllers and peripheral devices may also be provided, as shown in FIG. 14. An input controller 1403 represents an interface to a various input device(s) 1404, such as a keyboard, mouse, or stylus. There may also be a scanner controller 1405, which communicates with a scanner 1406. System 1400 may also include a storage controller 1407 for interfacing with one or more storage devices 1408 each of which includes a storage medium such as magnetic tape or disk, or an optical medium that might be used to record programs of instructions for operating systems, utilities and applications which may include embodiments of programs that implement various aspects of the present invention. The storage device(s) 1408 may also be used to store processed data or data to be processed in accordance with the invention. System 1400 may also include a display controller 1409 for providing an interface to a display device 1411, which may be a cathode ray tube (CRT), a thin film transistor (TFT) display, or other types of display. System 1400 may also include a printer controller 1412 for communicating with a printer 1413. A communications controller 1414 may interface with one or more communication devices 1415, which enables system 1400 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 1416, which may represent more than one physical bus. However, various system components may or may not be in physical proximity to one another. For example, input data and/or output data may be remotely transmitted from one physical location to another. In addition, programs that implement various aspects of this invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application-specific integrated circuits (ASICs), programmable logic devices (PLDs), flash memory devices, and ROM and RAM devices.

Embodiments of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that has computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application-specific integrated circuits (ASICs), programmable logic devices (PLDs), flash memory devices, and ROM and RAM devices. Examples of computer code include machine code, such as produced by a compiler, and files containing higher-level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. A non-transitory tangible computer-readable medium or media comprising one or more sequences of instructions which, when executed by one or more processors, causes steps to be performed comprising:
receiving data obtained from a urinary system test for a patient, the diagnostic engine having been trained to associate a data obtained from the urinary system test with one or more lower urinary tract dysfunction;
extracting one or more features from the received data;
identifying one or more urodynamic parameters of the patient, based on the one or more extracted features; and
generating an output that includes information of one or more lower urinary tract dysfunction associated with the one or more identified urodynamic parameters.

2. The non-transitory tangible computer-readable medium or media of claim 1, wherein the urinary system test includes a uroflowmetry and the received data includes at least one of an electromyography (EMG) signal that represents an electrical activity produced by a urethral sphincter or pelvic floor muscles of the patient, a plot of flow rate of the patient and a plot of voided volume of the patient.

3. The non-transitory tangible computer-readable medium or media of claim 2, wherein the one or more extracted features include at least one of a maximum flow rate in the plot of flow rate, a voiding time and a number of local maxima in the plot of flow rate.

4. The non-transitory tangible computer-readable medium or media of claim 1, wherein the urinary system test includes a cystometry and the received data includes at least one of an electromyography (EMG) signal that represents an electrical activity produced by a urethral sphincter or pelvic floor muscles of the patient, a plot of volume of fluid filled in a bladder of the patient, a plot of intra-abdominal pressure, a plot of intra-vesical pressure and a volume of post-void residual (PVR) in the bladder.

5. The non-transitory tangible computer-readable medium or media of claim 4, wherein the steps further comprise:
generating a plot of a detrusor pressure versus the volume of fluid filled in the bladder of the patient, the detrusor pressure being the intra-vesical pressure subtracted by the intra-abdominal pressure,
wherein the one or more extracted features include at least one of a slope of the detrusor pressure in the plot of the detrusor pressure during a storage phase of the cystometry, the volume of fluid when the patient feels a strong desire to void, the volume of fluid when an involuntary detrusor contraction occurs, and a number of involuntary detrusor contractions during the storage phase.

6. The non-transitory tangible computer-readable medium or media of claim 4, wherein the steps further comprise:
generating a plot of a detrusor pressure versus the flow rate, the detrusor pressure being the intra-vesical pressure subtracted by the intra-abdominal pressure,
wherein the one or more extracted features include at least one of a pattern of the plot of the detrusor pressure versus the flow rate, a value of the detrusor pressure when the patient starts voiding, a value of the detrusor pressure when the patient stops voiding, and a maximum value of the flow rate.

7. The non-transitory tangible computer-readable medium or media of claim 4, wherein the steps further comprise:
generating a plot of a detrusor pressure versus the volume of fluid in the bladder of the patient, the detrusor pressure being the intra-vesical pressure subtracted by the intra-abdominal pressure,
wherein the one or more extracted features include an average value of the detrusor pressure in the plot of the detrusor pressure during a voiding phase of the cystometry.

8. The non-transitory tangible computer-readable medium or media of claim 1, wherein the urinary system test includes a urethral pressure profile measurement and the received data includes at least one of a plot of intra-vesical pressure, and a plot of urethral pressure measured by a pressure sensor while the pressure sensor travels along a urethra of the patient.

9. The non-transitory tangible computer-readable medium or media of claim 8, wherein the steps further comprise:
generating a plot of a urethral pressure profile, the urethral pressure being a difference between the pressure measured by the urethral pressure sensor and the intra-vesical pressure,
wherein the one or more extracted features include a maximum value of the urethral pressure in the plot of the urethral pressure.

10. The non-transitory tangible computer-readable medium or media of claim 1, wherein the urinary system test includes a leak point pressure measurement and the received data includes at least one of an electromyography (EMG) signal that represents an electrical activity produced by an urethral sphincter or pelvic floor muscles of the patient, a plot of intra-abdominal pressure, and a plot of intra-vesical pressure and wherein the one or more extracted features include at least one of a Valsalva leak point pressure (VLPP) and a cough-induced leak point pressure (CLPP) and a detrusor leak point pressure (DLPP).

11. The non-transitory tangible computer-readable medium or media of claim 1, wherein the steps further comprise:
auto-detecting and screening of one or more artifacts in fluoroscopic urodynamic study results; and
listing the one or more artifacts.

12. A system for diagnosing an illness of a patient, comprising:
one or more processors; and
a diagnostic engine communicatively coupled to one or more processors, the diagnostic engine having been trained to associate data obtained from a urinary system test with one or more lower urinary tract dysfunction and configured to perform the steps of:
receiving data obtained from a urinary system test for a patient;
extracting one or more features from the received data;
identifying one or more urodynamic parameters of the patient, based on the one or more extracted features; and
generating an output that includes information of one or more lower urinary tract dysfunction of the patient associated with the one or more identified parameters.

13. The system of claim 12, wherein the one or more extracted features include one or more of a maximum flow rate in the plot of flow rate, a voiding time and a number of local maxima in the plot of flow rate.

14. The system of claim 13, wherein the urinary system test includes a uroflowmetry and the received data includes at least one of an electromyography (EMG) signal that represents an electrical activity produced by a urethral sphincter or pelvic floor muscles of the patient, a plot of flow rate of the patient and a plot of volume of fluid voided by the patient.

15. The system of claim 12, wherein the urinary system test includes a cystometry and the received data includes at least one of an electromyography (EMG) signal that represents an electrical activity produced by an urethral sphincter or pelvic floor muscles of the patient, a plot of volume of fluid filled in a bladder of the patient, a plot of intra-abdominal pressure, a plot of intra-vesical pressure and a volume of post-void residual (PVR) in the bladder.

16. The system of claim 15, wherein the diagnostic engine is configured to perform an additional step of:
generating a plot of a detrusor pressure versus the volume of fluid filled in the bladder of the patient, the detrusor pressure being the intra-vesical pressure subtracted by the intra-abdominal pressure,
wherein the one or more extracted features include one or more of a slope of the detrusor pressure in the plot of the detrusor pressure during a storage phase of the cystometry, the volume of fluid when the patient feels a strong desire to void, the volume of fluid when an involuntary detrusor contraction occurs, and a number of involuntary detrusor contractions during the storage phase.

17. The system of claim 15, wherein the diagnostic engine is configured to perform an additional step of:
generating a plot of a detrusor pressure versus the flow rate, the detrusor pressure being the intra-vesical pressure subtracted by the intra-abdominal pressure,
wherein the one or more extracted features include one or more of a pattern of the plot of the detrusor pressure versus the flow rate, a value of the detrusor pressure when the patient starts voiding, a value of the detrusor pressure when the patient stops voiding, and a maximum value of the flow rate.

18. The system of claim 15, wherein the diagnostic engine is configured to perform an additional step of:
generating a plot of a detrusor pressure versus the volume of fluid in the bladder of the patient, the detrusor pressure being the intra-vesical pressure subtracted by the intra-abdominal pressure,
wherein the one or more extracted features include an average value of the detrusor pressure in the plot of the detrusor pressure during a voiding phase of the cystometry.

19. The system of claim 12, wherein the urinary system test includes a urethral pressure profile measurement and the received data includes at least one of a plot of intra-vesical pressure, and a plot of urethral pressure measured by a pressure sensor while the pressure sensor travels along a urethra of the patient.

20. The system of claim 19, wherein the diagnostic engine is configured to perform an additional step of:
generating a plot of a urethral pressure, the urethral pressure profile being a difference between the urethral pressure measured by the pressure sensor and the intra-vesical pressure,
wherein the one or more extracted features include a maximum value of the urethral pressure in the plot of the urethral pressure.

21. The system of claim 12, wherein the urinary system test includes a leak point measurement and the received data includes at least one of an electromyography (EMG) signal that represents an electrical activity produced by an urethral sphincter or pelvic floor muscles of the patient, a plot of intra-abdominal pressure, and a plot of intra-vesical pressure and wherein the one or more extracted features include at least one of a Valsalva leak point pressure (VLPP) and a cough-induced leak point pressure (CLPP) and a detrusor leak point pressure (DLPP).

22. The system of claim 12, wherein the diagnostic engine is configured to perform additional steps of:
auto-detecting and screening of one or more artifacts in the fluoroscopic urodynamic study results; and
listing the one or more artifacts.

23. The system of claim 12, wherein the urinary system test includes a leak point measurement and the received data includes at least one of an electromyography (EMG) signal that represents an electrical activity produced by an urethral sphincter or pelvic floor muscles of the patient, a plot of intra-abdominal pressure, and a plot of intra-vesical pressure and wherein the one or more extracted features include at least one of a Valsalva leak point pressure (VLPP) and a cough-induced leak point pressure (CLPP).
